(19) United States
(12) Patent Application Publication
(10) Pub. No.: US 2022/0119441 A1
(43) Pub. Date: Apr. 21, 2022

(54) METHOD FOR PREPARING DRUG-LINKER MC-MMAF FOR ANTIBODY DRUG CONJUGATE, AND INTERMEDIATES THEREIN

(71) Applicant: LEVENA BIOPHARMA CO., LTD., Suzhou (CN)

(72) Inventors: Zhe Xu, Suzhou (CN); Haihong Li, Suzhou (CN); Maojun Guo, Suzhou (CN); Hui Li, Suzhou (CN)

(21) Appl. No.: 17/426,634

(22) PCT Filed: Jun. 26, 2019
(86) PCT No.: PCT/CN2019/092950

§ 371 Date: Jul. 29, 2021

(30) Foreign Application Priority Data
Mar. 8, 2019 (CN) 201910178142.X

(51) Int. Cl. C07K 1/06; A61K 47/68; A61K 47/65; C07K 5/02; C07K 5/06

(52) U.S. Cl. CPC C07K 1/062; A61K 47/6803; C07K 5/06; C07K 5/0205; A61K 47/65

(57) ABSTRACT

The disclosure provides a method for preparing drug-linker MC-MMAF for antibody drug conjugates and intermediates therein. The preparation method of the present disclosure improves the reactivity of the N-terminal, thereby effectively controlling the occurrence of racemization; does not directly use the toxin MMAF, but uses fragmented peptides with lower toxicity, which minimizes the operational difficulty in scale-up production; no reverse phase is required and it is easy to prepare and operate.

### TECHNICAL FIELD

The present disclosure relates to the field of organic synthesis, in particular to a method for preparing drug-linker MC-MMAF for antibody drug conjugates and intermediates therein.

### BACKGROUND

Antibody drug conjugate (ADC) is a new type of anti-tumor drug. Its principle is to connect cytotoxin to antibody. Through antibody recognition of specific antigen on the surface of cancer cell, it enters the cancer cells through endocytosis, thereby transporting cytotoxins to the target, achieve the purpose of targeted treatment of malignant tumors. Compared with traditional small-molecule anti-tumor drugs, ADC is more specific and effective because it can rely on the target recognition of antibodies and the high activity of toxins.

ADC includes three different components, namely antibody, linker and cytotoxin. The antibody achieves targeting, and the linker ensures the stability of the ADC during blood transport, and after reaching the target point, the toxin exerts a killing effect on cancer cells. According to different mechanisms of action, toxins appropriate for ADC are classified into microtubule inhibitors, DNA damaging agents, RNA polymerase inhibitors, and et al. At present, the toxins used by ADCs commercial available and in clinical trials are mainly microtubule inhibitors, mainly including dolastatin-based compounds, such as MMAE, MMAF and MMAD, and maytansine-based (Maytansine-based) designed compounds, such as DM1 and DM4. In terms of linkers, the main applications are non-cleavable types, such as valine-citrulline (Valine-Citriline) and cyclohexyl carboxylic acid (MCC). After lysosomal hydrolysis, the drug is still active, and is connected to a certain amino acid residue through link area.

There are many ways to form antibody-drug conjugates. Either the amino or sulfhydryl group on the antibody and the drug linker can be chemically coupled, or the antibody can be modified. After a specific functional group is introduced on the antibody, it can be coupled with the drug linker for chemical reaction or enzyme-catalyzed reaction coupling. The structure of the drug-linker MC-MMAF involved in the present disclosure is shown below.

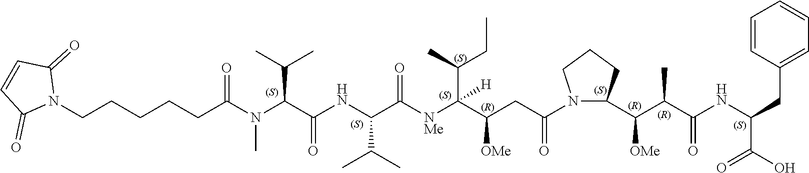

The synthesis route of MC-MMAF currently reported in the literature is to use the toxin MMAF and MC-hex-Acid (1-maleimido n-hexanoic acid) to perform a dehydration reaction to obtain MC-MMAF. The structure of MMAF is:

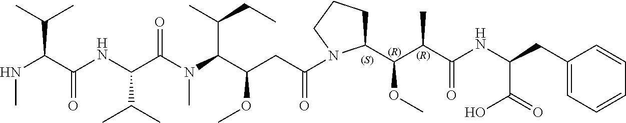

The synthesis scheme reported in the literature is:

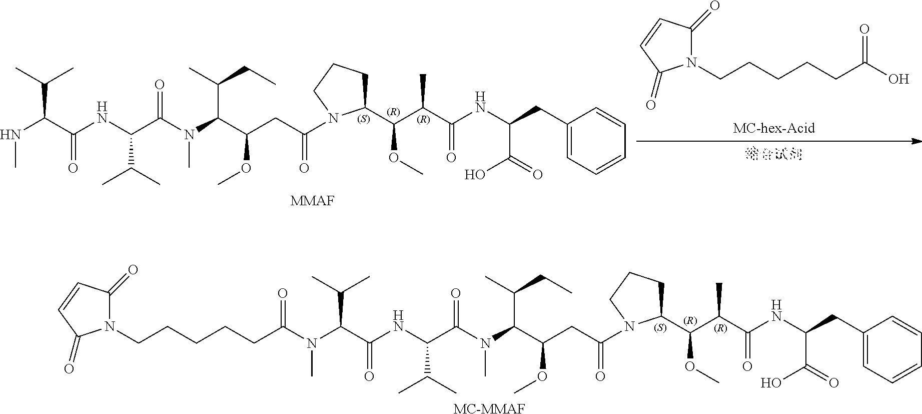

The N-terminal valine of this route of MMAF has a methyl group on the N, which is sterically hindered. In this case, the reaction speed of connecting 1-maleimido-n-hexanoic acid to MMAF will be slower. Even if a different amide condensing agent is used, it will cause racemization of the chiral carbon linked to the phenylpropionamide group of MMAF. This route is used for the synthesis of MC-MMAF of less than 1 g, and finally high-pressure reverse phase preparation is used to remove isomeric impurities, and the yield is less than 50%.

This reaction route shows certain defects during scale-up production, such as: 1. Because the condensing agent will activate the carboxyl group on MMAF at the same time, this method will cause 30-50% racemization, forming difficult-to-remove isomer impurities, affecting yield; 2. Due to the aforementioned steric hindrance, the reaction time is long, and there are many impurities, which cause difficulties in the post-treatment and purification of the reaction; 3. The final product requires high-pressure reverse phase preparation to remove isomers, which increases operating costs; 4. Direct using the toxin MMAF as a raw material, it is necessary to do a good protection in scale-up of synthesis operations, and the selection of protective equipment will bring obstacles to the production operation.

### SUMMARY

On the one hand, in view of the defects in the prior art, the present disclosure provides a method for synthesizing MC-MMAF. The key to the reaction is to use a compound of structural formula

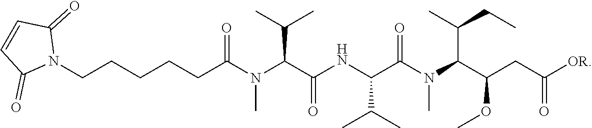

to condense with the structural fragment peptide Dap-Phe-OH of MMAF to directly obtain MC-MMAF or a salt thereof, R is selected from a group consisting of hydrogen, succinimidyl, pentafluorophenyl, p-nitrophenyl, phthalamide, and a mixture thereof.

The chemical structure of Dap-Phe-OH is as follows:

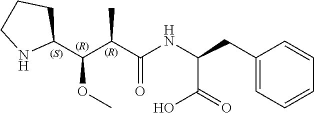

The above-mentioned objects of the present disclosure are achieved by the following technical solutions.

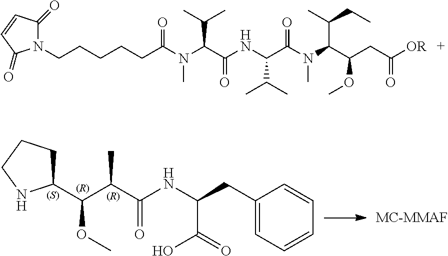

The synthesis method includes the following steps: 1) Dissolve the compound

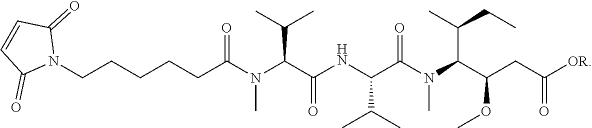

in an appropriate solvent and an amide condensation reaction occurs with Dap-Phe-OH to obtain MC-MMAF.

Preferably, in step 1), the appropriate solvent is selected from a group consisting of dichloromethane, dimethyl sulfoxide, N,N-dimethylformamide, N,N-dimethylacetamide, tetrahydrofuran, 1,4-dioxane, 2-methyltetrahydrofuran and a mixture thereof, more preferably, the appropriate solvent is selected from a group consisting of dichloromethane and N,N-dimethylformamide and a mixture thereof.

Preferably, in step 1), if R is hydrogen, reagent N is added in the presence of reagent M, which is selected from a group consisting of DCC, DCEP, EDC, DIC, HATU, HBTU, HBPIPU, HBPyU, HSPyU, HCTU, HOTU, HOTT, HSTU, HDMA, TATU, TBTU, TCTU, TCFH, TDBTU, TOTU, TOTT, TPTU, TFFH, BTFFH, TNTU, TSTU, COMU, T3P, BOP, PyBOP, PyBrOP, PyClOP, Brop, PyAOP, PyCIU, CDI, TPSI, TSTU, DEPBT, DMTMM, EEDQ, CIP, CIB, DMC, HOBt, EDCI and a mixture thereof, more preferably, the reagent M is selected from a group consisting of EDCI, EDC, DIC, HOAt, HOBt and a mixture thereof, further preferably, the reagent M is a mixture of EDCI, EDC, DIC, HOAt, HOBt, and a mixture thereof, most preferably, the reagent M is a mixture of EDCI, EDC, DIC, HOAt and HOBt. The reagent N is selected from a group consisting of triethylamine, diisopropylethylamine (DIEA), pyridine, N,N-dimethyl-4-pyridine, and is preferably diisopropylethylamine (DIEA). The reaction temperature of the reaction is 20° C. subzero to 40° C., preferably 10° C. subzero to 25° C.

Preferably, in step 1), if R is selected from a group consisting of perimido group, pentafluorophenyl group, p-nitrophenyl group, phthalamide group and a mixture thereof, in the presence of reagent P, it reacts with Dap-Phe-OH to obtain MC-MMAF. The reagent P is selected from a group consisting of triethylamine, diisopropylethylamine (DIEA), pyridine, N,N-dimethyl-4-pyridine, sodium carbonate, sodium bicarbonate, potassium carbonate, potassium bicarbonate, lithium carbonate, lithium bicarbonate and a mixture thereof, preferably sodium carbonate, or diisopropylethylamine (DIEA). The reaction temperature is 0° C. to 100° C., preferably 15° C. to 50° C.

Preferably, in step 1), it further comprises a step of separating MC-MMAF from the reaction liquid after the reaction is completed.

Preferably, the separation comprises evaporating the solvent under reduced pressure, and then purifying or recrystallizing by medium pressure chromatography to obtain MC-MMAF.

The preparation method of the present disclosure improves the reactivity of the N-terminal, thereby effectively controlling the occurrence of racemization; does not directly use the toxin MMAF, but uses fragmented peptides with lower toxicity, which minimizes the operational difficulty in scale-up production; no reverse phase preparation is required, it is easy to prepare and operate. As mentioned above, the method minimizes the difficulty of operation, makes the quality standard easier to control, and can be applied to the preparation of one hundred grams.

On the other hand, this patent also provides an intermediate compound for the synthesis of MC-MMAF, the structural formula

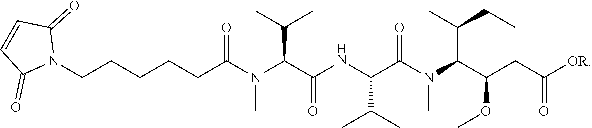

of which is, wherein R is selected from a group consisting of hydrogen, succinimidyl, pentafluorophenyl, p-nitrophenyl, phthalamide and a mixture thereof. It is preferably the following compound, as shown in Table 1:

TABLE 1

| No. | Structural formula |
|---|---|
| Compond 1 | 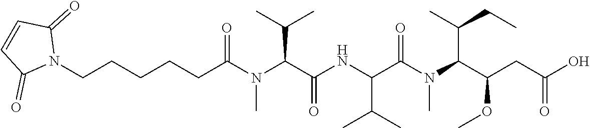 [SMILES: CC[C@H](C)[C@@H]([C@@H](CC(=O)O)OC)N(C)C(=O)C(NC(=O)[C@H](C(C)C)N(C)C(=O)CCCCCN1C(=O)C=CC1=O)C(C)C] |
| Compoud 2 | 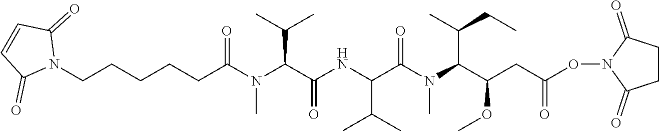 [SMILES: CC[C@H](C)[C@@H]([C@@H](CC(=O)ON1C(=O)CCC1=O)OC)N(C)C(=O)C(NC(=O)[C@H](C(C)C)N(C)C(=O)CCCCCN1C(=O)C=CC1=O)C(C)C] |

In another aspect, the present disclosure also provides a method of synthesis

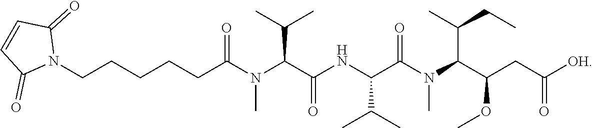 [SMILES: CC[C@H](C)[C@@H]([C@@H](CC(=O)O)OC)N(C)C(=O)[C@@H](NC(=O)[C@H](C(C)C)N(C)C(=O)CCCCCN1C(=O)C=CC1=O)C(C)C]

The synthesis steps are as follows:

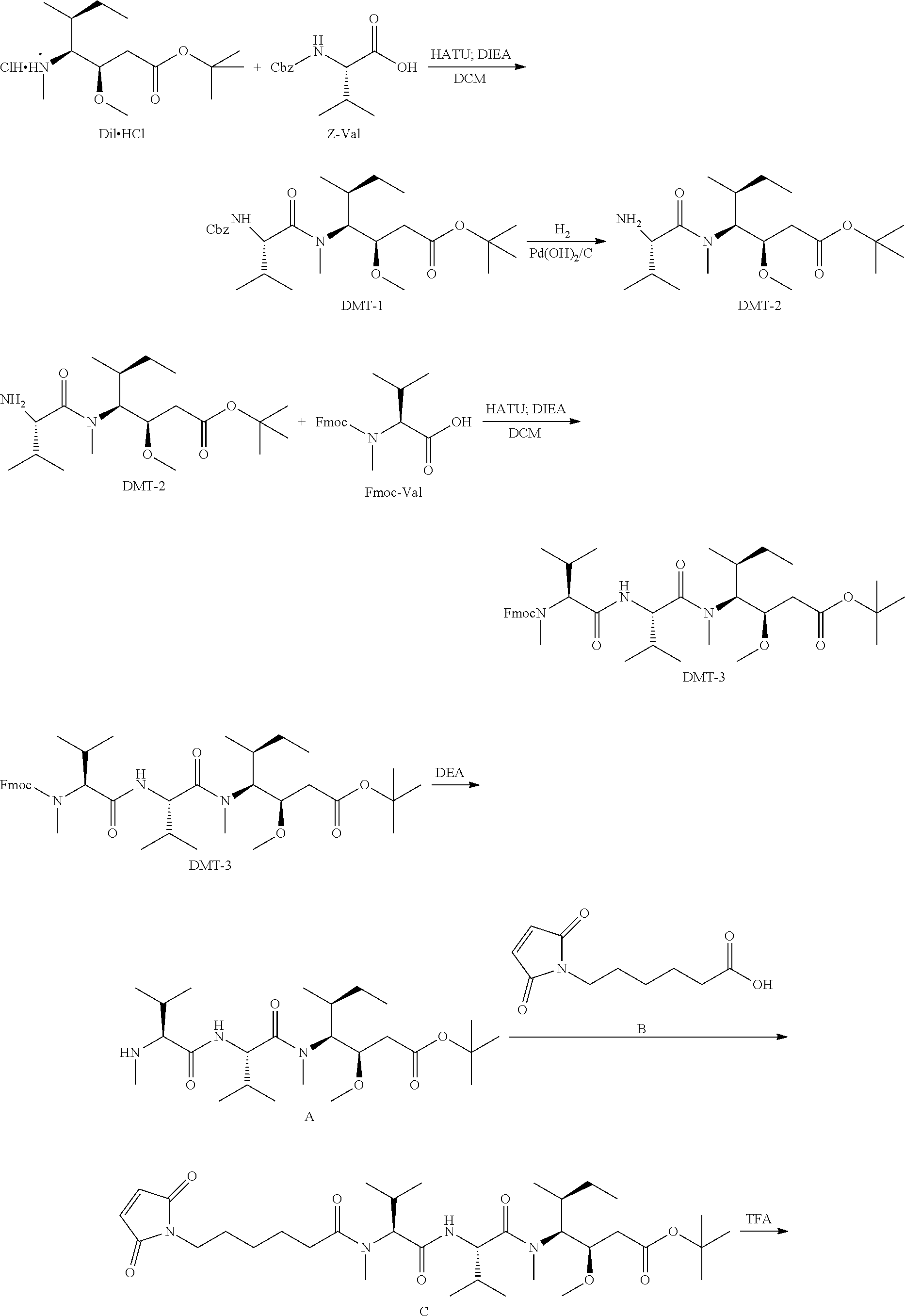

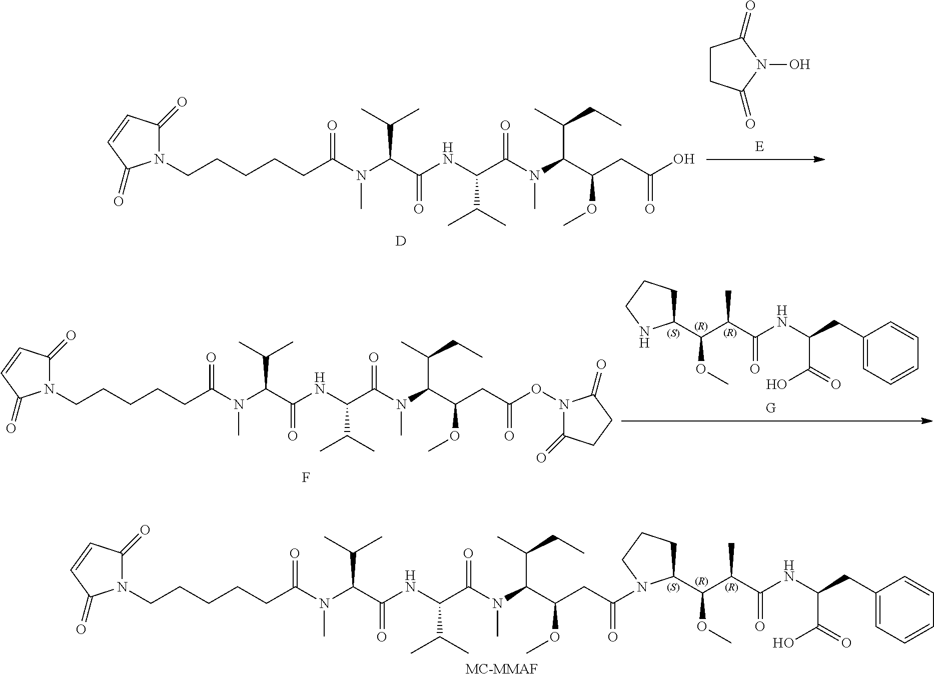

This route contains synthesizing of important intermediate D, and there is no report on the synthesis method of this compound before.

Compound D cannot be synthesized with polypeptide X without a protective group. In the experiment, a self-condensed product of polypeptide X was obtained. That is, the synthesis route shown below cannot directly synthesize compound D.

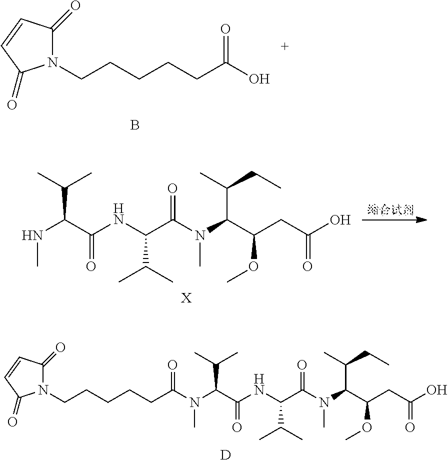

In the technical scheme employed in the present disclosure, compound C is first synthesized using a polypeptide with a protective group, and then compound D is obtained by deprotecting the protective group under acidic conditions. However, it was found in experiments that compound C and compound D are very unstable under acidic conditions, and the amide bond in the middle of the molecule will be broken, resulting in a very low yield. After further research, it is found that increasing the concentration of acid will greatly increase the speed of deprotection, but not much for the speed of side reactions. The concentration of trifluoroacetic acid (this concentration refers to the concentration of trifluoroacetic acid in the reaction solution in a solvent such as dichloromethane) ranges from 30% to 50%, preferably 35%, the deprotection reaction will be completed quickly and then quenched immediately. Finally, the yield of compound D can be increased from 5% to 50%.

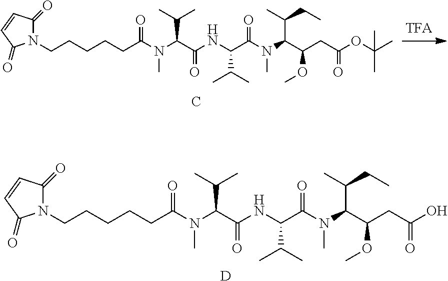

side effects:

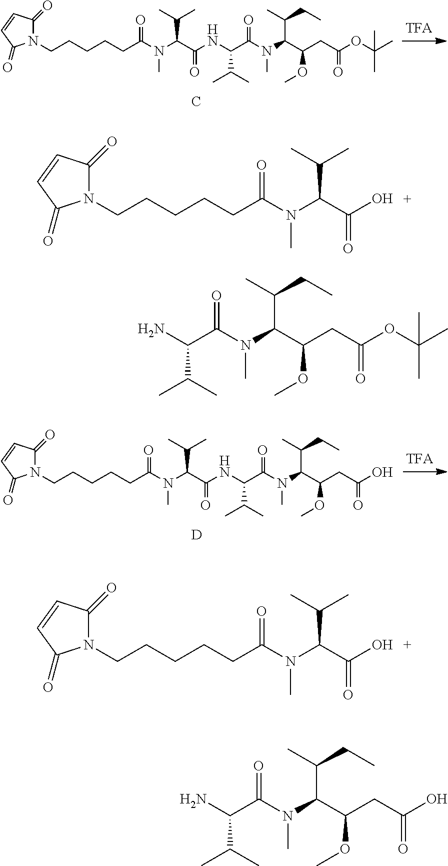

By selecting acid reagents and controlling the reaction conditions of acid concentration, the yield of compound D is greatly improved, making this route possible to be applied to production.

The present disclosure abandons the existing MMAF synthesis route and regards MC-MMAF as a whole to synthesize. The biggest problem is that MC linkers are fragments with relatively high reactivity. Connecting MC in advance will increase the difficulty of synthesis. Those skilled in the art would not think of this route. Through many studies, we have solved the problem of instability in the synthesis of the MC fragment compound introduced in advance, so that this overall synthetic route can be realized.

As used in this article, the definitions of commonly used organic abbreviations and their corresponding CAS numbers are shown in Table 2:

TABLE 2

| Abbreviation | Definition | CAS No. |
|---|---|---|
| BrOP | Bromotris(dimethylamino)phosphorus hexafluorophosphate | 50296-37-2 |
| DBU | 1,8-diazabicyclo[5.4.0]undec-7-ene | 6674-22-2 |
| DECP | Diethyl cyanophosphate | 2942-58-7 |
| DIEA | N,N-Diisopropylethylamine | 7087-68-5 |
| DMT | Dimethyl Val-Val-Dil-OH | 133120-89-5 |
| HOSu | N-hydroxysuccinimide | 6066-82-6 |
| TEA | Triethylamine | 121-44-8 |
| DCC | N,N′-Dicyclohexylcarbodiimide | 538-75-0 |
| EDCI | 1-(3-Dimethylaminopropyl)-3-ethylcarbodiimide hydrochloride | 7084-11-9 |
| DIC | N,N′-Diisopropylcarbodiimide | 693-13-0 |
| HATU | 2-(7-Azobertzotriazole)-N,N,N′,N′-tetramethylurea | 148893-10-1 |
| | hexafluorophosphate | |
| HBTU | Benzotriazole-N,N,N′,N′-tetramethylurea hexafluorophosphate | 94790-37-1 |
| HBPIPU | (Benzotriazol-1-yloxy)dipiperidine carbohexafluorophosphate | 206752-41-2 |
| HBPyU | O-(benzotriazol-1-yl)-N,N,N′,N′-dipyrrolylurea | 105379-24-6 |
| | hexafluorophosphate | |
| HSPyU | Dipyrrolidinyl (N-succinimidyloxy) hexafluorophosphate | 207683-26-9 |
| HCTU | 6-Chlorobenzotriazole-1,1,3,3-tetramethylurea | 330645-87-9 |
| | hexafluorophosphate | |
| HOTU | O-[(Ethoxycarbonyl)cyanomethylamine]-N,N,N′,N′- | 333717-40-1 |
| | tetramethylthiourea hexafluorophosphate | |
| HOTT | N,N,N′,N′-Tetramethyl-S-(1-oxo-2-pyridyl)thiourea | 212333-72-7 |
| | hexafluorophosphate | |
| HSTU | N,N,N′,N′-tetramethylurea-O-(N-succinimidyl) | 265651-18-1 |
| | hexafluorophosphate | |
| HDMA | 1- [(Dimethylamino)(morpholine)methyl]-3-oxo-1H-[1,2,3] | 958029-37-3 |
| | triazole[4,5-b]pyridine 3-hexafluorophosphate | |
| TATU | 2-(7-Azabenzotriazole)-N,N,N′,N′-tetramethylurea | 873798-09-5 |
| | tetrafluoroborate | |
| TBTU | O-benzotriazole-N,N,N′,N′-tetramethylurea tetrafluoroborate | 125700-67-6 |
| TCTU | O-(6-Chloro-1H-benzotriazol-1-yl)-N,N,N′,N′-tetramethylurea | 330641-16-2 |
| | tetrafluoroborate | |
| TCFH | N,N,N′,N′-Tetramethylchloroformamidine hexafluorophosphate | 94790-35-9 |
| TDBTU | N,N,N′,N′-tetramethyl-O-(4-carbonyl-3,4-dihydro-1,2,3- | 125700-69-8 |
| | benzotriazin-3-yl)urea tetrafluoroborate | |
| TOTU | O-[(Ethoxycarbonyl)cyanomethylamine]-N,N,N′,N′- | 136849-72-4 |
| | tetramethylthiourea tetrafluoroboron | |
| TOTT | 2-(1-Pyridin-2-yl oxide)-1,1,3,3-Tetramethylisothiourea | 255825-38-8 |
| | tetrafluoroborate | |
| TPTU | 2-(2-pyridone-1-yl)-1,1,3,3-tetramethylurea tetrafluoroborate | 125700-71-2 |
| TFFH | Fluoro-N,N,N′,N′-tetramethylurea hexafluorophosphate | 164298-23-1 |
| BTFFH | N,N,N′,N′-bis(tetramethylene)fluoroformamidine | 164298-25-3 |
| | hexafluorophosphate | |
| TNTU | 2-(Endo-5-norbornene-2,3-dicarboximide)-1,1,3,3-tetramethylurea | 125700-73-4 |
| | tetrafluoroborate | |
| TSTU | 2-succinimidyl-1,1,3,3-tetramethylurea tetrafluoroborate | 105832-38-0 |
| COMU | cycluron | 2163-69-1 |
| T3P | Propyl phosphate tricyclic anhydride | 68957-94-8 |
| BOP | 1H-benzotriazol-1-yloxotris(dimethylamino)phosphonium | 56602-33-6 |
| | hexafluorophosphate | |
| PyBOP | 1H-benzotriazol-1-yloxytripyrrolidinyl hexafluorophosphate | 128625-52-5 |
| PyBrOP | Tripyrrolidinylphosphonium bromide hexafluorophosphate | 132705-51-2 |
| PyClOP | Chlorotripyrrolidinyl hexafluorophosphate | 133894-48-1 |
| BrOP | Bromotris(dimethylamino)phosphonium hexafluorophosphate | 50296-37-2 |
| PyAOP | (3H-1,2,3-Triazolo[4,5-b]pyridin-3-oxy)tris-1-pyrrolidinyl | 156311-83-0 |
| | hexafluorophosphate | |
| PyCIU | 1-(Chloro-1-pyrrolidinylmethylene)pyrrolidine | 135540-11-3 |
| | hexafluorophosphate | |
| CDI | N,N′-Carbonyl Diimidazole | 530-62-1 |
| TsIm | 1-p-toluenesalfonyl imidazole | 2232-08-8 |
| TPSI | 1-(2,4,6-triisopropylphenylsulfonyl)imidazole | 50257-40-4 |
| TSTU | 2-succinimidyl-1,1,3,3-tetramethylurea tetrafluoroborate | 105832-38-0 |
| DEPBT | 3-(diethoxy o-acyloxy)-1,2,3-benzotriazin-4-one | 165534-43-0 |
| DMTMM | 4-(4,6-Dimethoxytriazin-2-yl)-4-methylmorpholine | 3945-69-5 |
| | hydrochloride | |
| EEDQ | 2-ethoxy-1-ethoxycarbonyl-1,2-dihydroquinoline | 16357-59-8 |
| CIP | 2-chloro-1,3-dimethylimidazolium hexafluorophosphate | 101385-69-7 |
| CIB | 2-chloro-1,3-dimethylimidazolium tetrafluoroborate | 153433-26-2 |
| DMC | 2-chloro-1,3-dimethylimidazolium chloride | 37091-73-9 |
| EDC | 1-(3-Dimethylaminopropyl)-3-ethylcarbodiimide hydrochloride | 7084-11-9 |
| DIC | N,N′-Diisopropylcarbodiimide | 693-13-0 |
| HOAt | N-hydroxy-7-azabenzotriazole | 39968-33-7 |
| HOBt | 1-hydroxybenzotriazole | 2592-95-2 |

### BRIEF DESCRIPTION OF THE DRAWINGS

FIG. 1 is a high performance liquid chromatography of DMT-3 synthesized in the present disclosure.

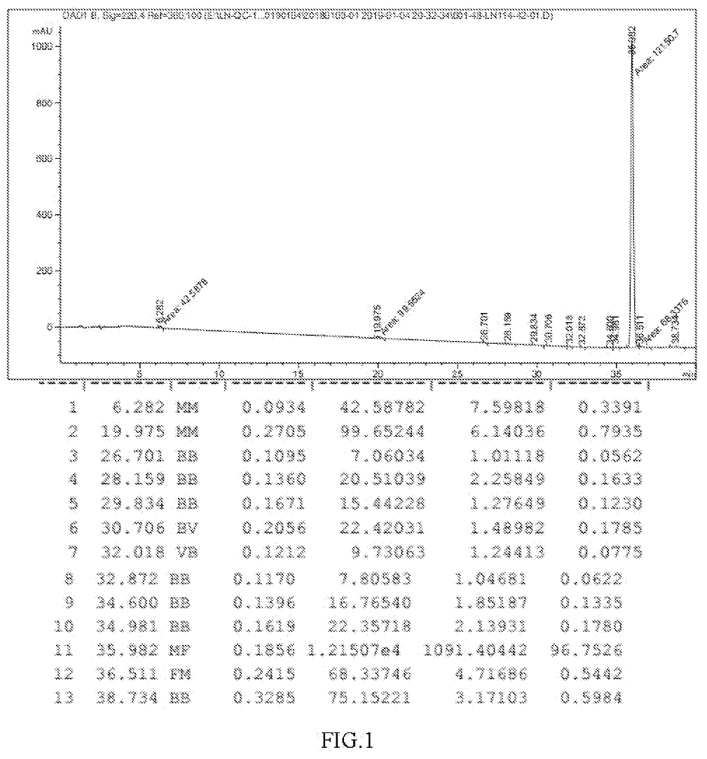

FIG. 2 is a liquid chromatography of compound A synthesized in the present disclosure.

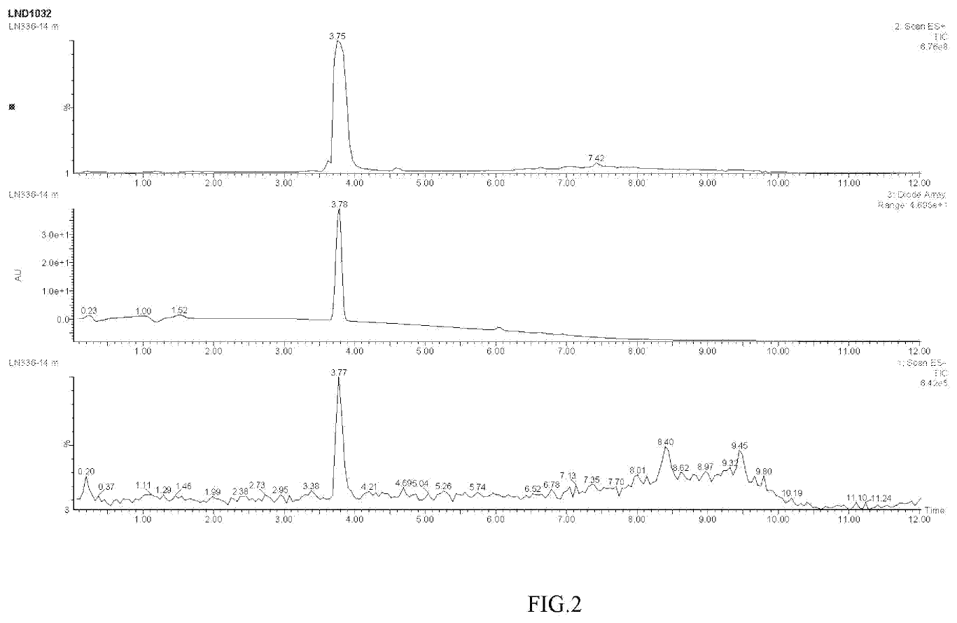

FIG. 3 is a mass spectrum of compound A synthesized in the present disclosure.

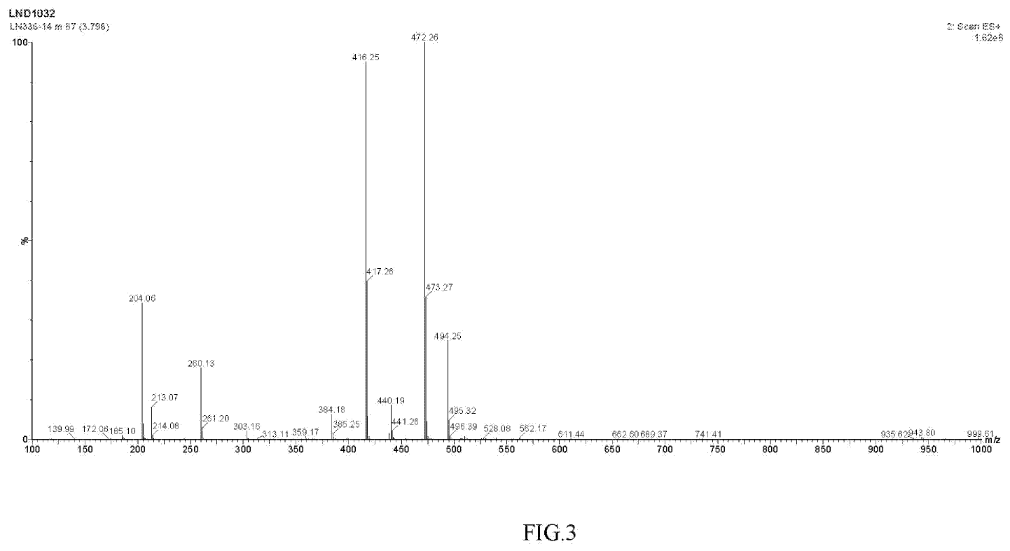

FIG. 4 is a liquid chromatography of compound C synthesized in the present disclosure.

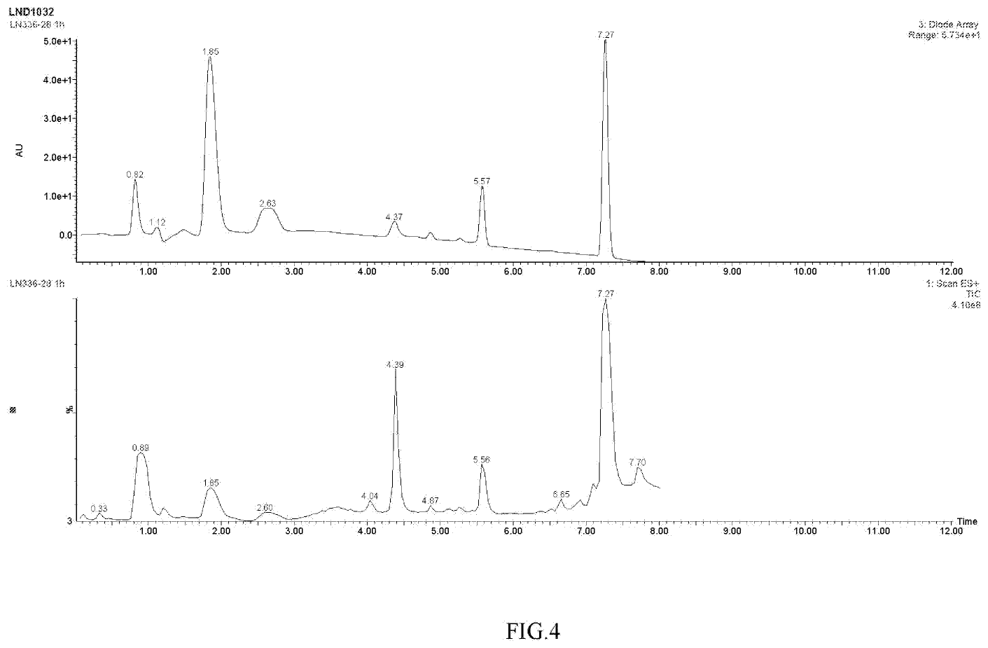

FIG. 5 is a mass spectrum of compound C synthesized in the present disclosure.

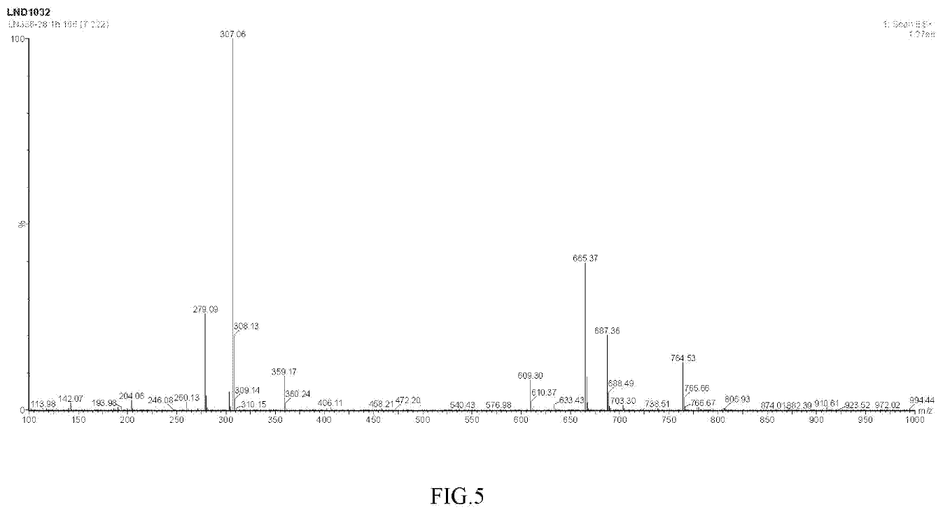

FIG. 6 is a liquid chromatography of compound D synthesized by the present disclosure.

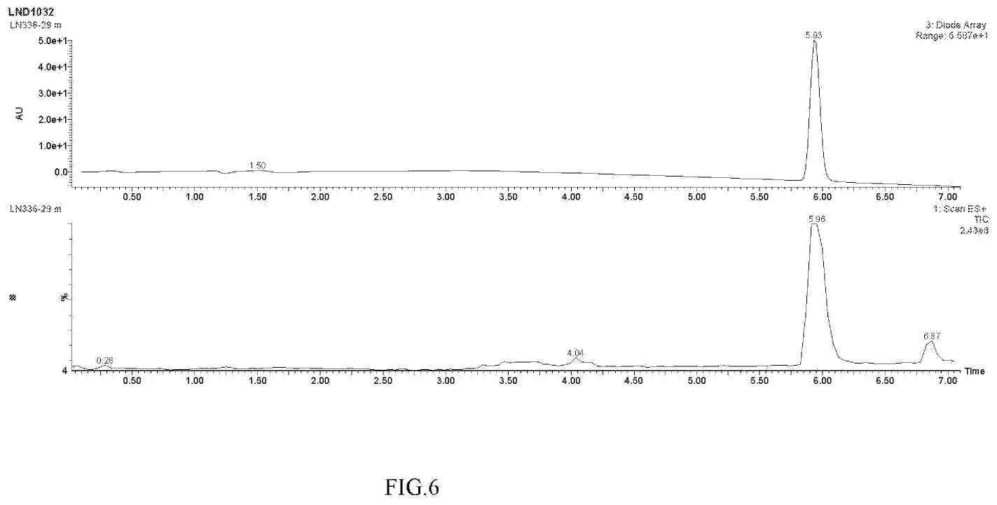

FIG. 7 is a mass spectrum of compound D synthesized by the present disclosure.

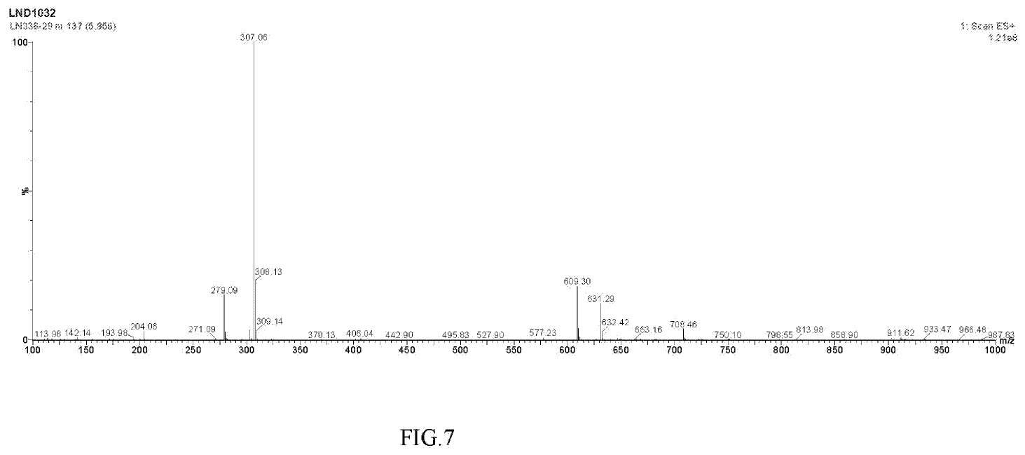

FIG. 8 is a liquid chromatography of compound F synthesized in the present disclosure.

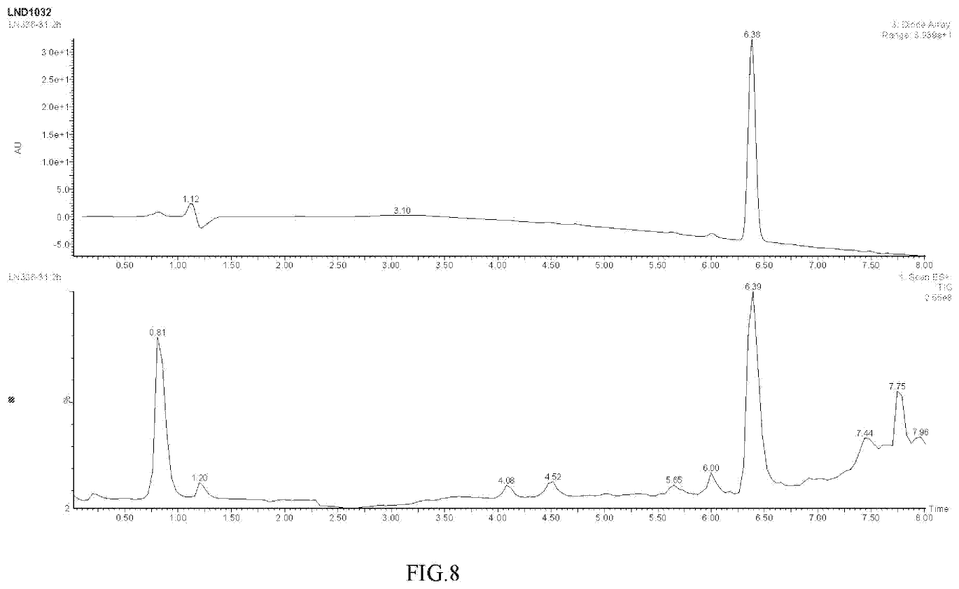

FIG. 9 is a mass spectrum of compound F synthesized in the present disclosure.

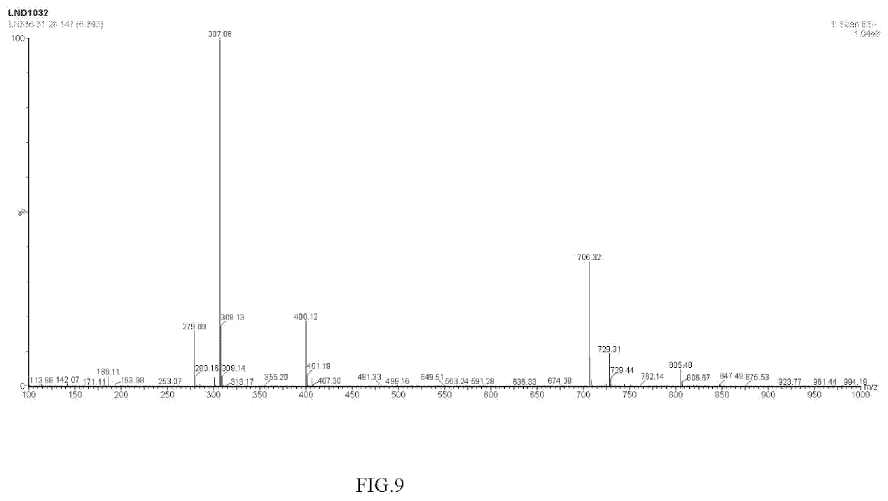

FIG. 10 is a liquid chromatography of the target product MC-MMAF synthesized by the present disclosure.

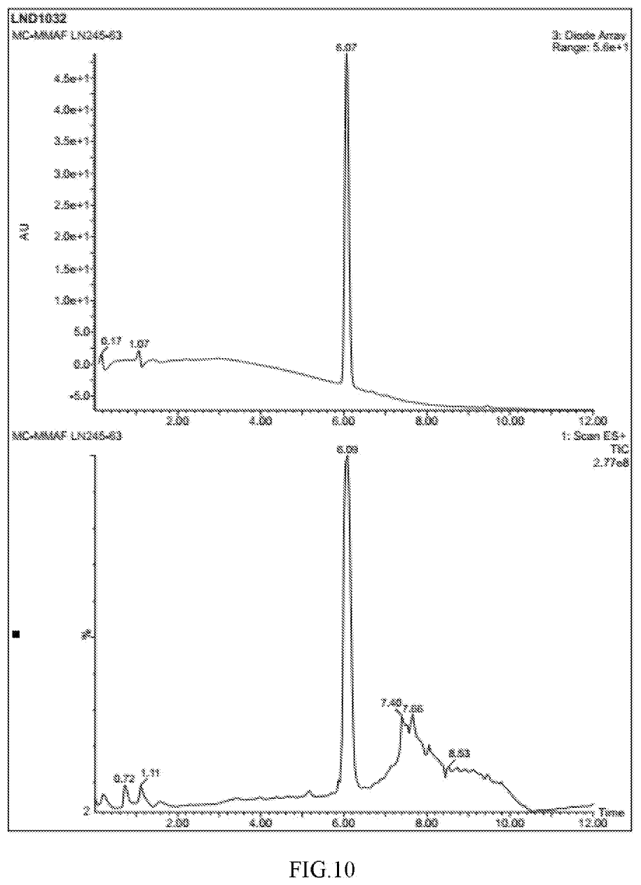

FIG. 11 is a mass spectrum of the target product MC-MMAF synthesized by the present disclosure.

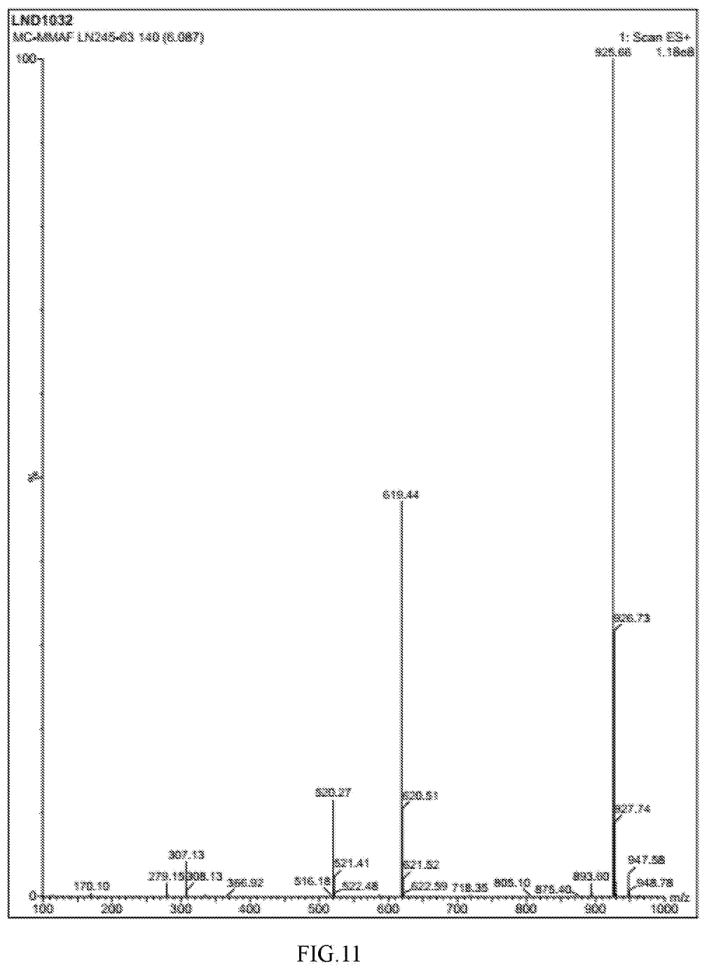

FIG. 12 is the NMR spectrum of the target product MC-MMAF synthesized by the present disclosure.

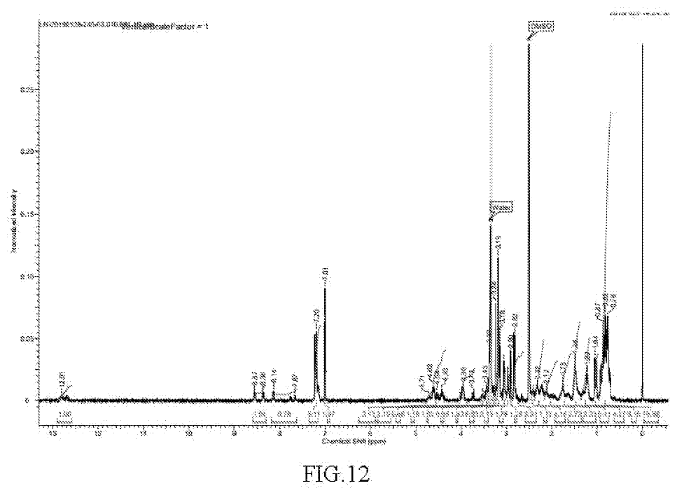

### DESCRIPTION OF THE EMBODIMENTS

The technical solution of the present disclosure will be further non-restrictively described in detail below with reference to specific embodiments. It should be pointed out that the following embodiments are only to illustrate the technical concept and features of the present disclosure, and their purpose is to enable those familiar with the technology to understand the content of the present disclosure and implement them accordingly, and cannot limit the protection scope of the present disclosure. All equivalent changes or modifications made according to the spirit of the present disclosure should be covered by the protection scope of the present disclosure.

LCMS means liquid-mass spectrometry detection method; HPLC means high-performance liquid chromatography detection.

The raw materials and reagents for each step of the reaction involved in the present disclosure can be purchased from the market or prepared according to the method of the present disclosure.

The present disclosure provides a method for synthesizing MC-MMAF, which includes the following steps:

1) Dissolve the compound

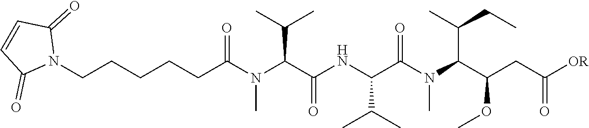

in an appropriate solvent selected from a group consisting of dichloromethane, dimethyl sulfoxide, N,N-dimethylformamide, N,N-dimethylacetamide, tetrahydrofuran, 1,4-dioxane, 2-methyltetrahydrofuran and a mixture thereof, will undergo amide condensation reaction with Dap-Phe-OH to obtain MC-MMAF. The structural formula of Dap-Phe-OH is

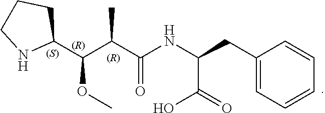

In step 1), if R is hydrogen, in the presence of reagent M, add reagent N, which is selected from a group consisting of DCC, DCEP, EDC, DIC, HATU, HBTU, HBPIPU, HBPyU, HSPyU, HCTU, HOTU, HOTT, HSTU, HDMA, TATU, TBTU, TCTU, TCFH, TDBTU, TOTU, TOTT, TPTU, TFFH, BTFFH, TNTU, TSTU, COMU, T3P, BOP, PyBOP, PyBrOP, PyClOP, Brop, PyAOP, PyCIU, CDI, TPSI, TSTU, DEPBT, DMTMM, EEDQ, CIP, CIB, DMC, HOBt, EDCI and a mixture thereof; more preferably, the reagent M is selected from a group consisting of EDCI, EDC, DIC, HOAt, HOBt and a mixture thereof; further preferably, the reagent M is a mixture of EDCI, EDC, DIC, HOAt and HOBt; most preferably, the reagent M is a mixture of EDCI and HOBt. The reagent N is selected from a group consisting of triethylamine, diisopropylethylamine (DIEA), pyridine, N,N-dimethyl-4-pyridine and a mixture thereof, and is preferably diisopropylethylamine (DIEA). The reaction temperature is 20° C. subzero to 40° C., preferably 10° C. subzero to 25° C.

In step 1), if R is selected from a group consisting of pyrimidyl, pentafluorophenyl, p-nitrophenyl, phthalamide and a mixture thereof, in the presence of reagent P, it will interact with Dap-Phe-OH reacts to obtain MC-MMAF. The reagent P is selected from a group consisting of triethylamine, diisopropylethylamine (DIEA), pyridine, N,N-dimethyl-4-pyridine, sodium carbonate, sodium bicarbonate, potassium carbonate, potassium bicarbonate, lithium carbonate, lithium bicarbonate and a mixture thereof, preferably sodium carbonate, or diisopropylethylamine (DIEA). The reaction temperature is 0° C. to 100° C., preferably 15° C. to 50° C.

In step 1), it also includes the step of separating MC-MMAF from the reaction solution after the reaction is completed. Preferably, the separation includes evaporating the solvent under reduced pressure, and then purifying or recrystallization by medium pressure chromatography to obtain MC-MMAF.

The present disclosure also provides a method of synthesizing

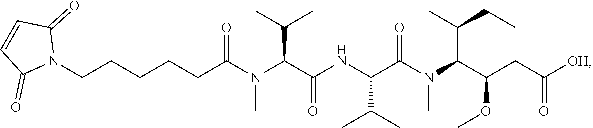 [SMILES: CC[C@H](C)[C@@H]([C@@H](CC(=O)O)OC)N(C)C(=O)[C@@H](NC(=O)[C@H](C(C)C)N(C)C(=O)CCCCCN1C(=O)C=CC1=O)C(C)C]

using a polypeptide with a protective group

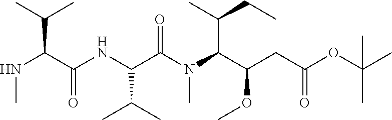

to reaction with

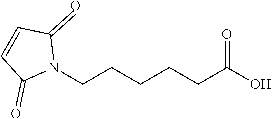

to first synthesize a compound

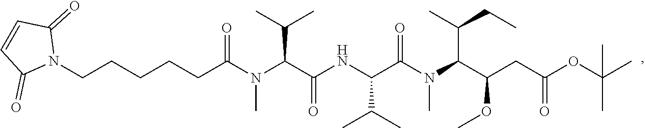 [SMILES: CC[C@H](C)[C@@H]([C@@H](CC(=O)OC(C)(C)C)OC)N(C)C(=O)[C@@H](NC(=O)[C@H](C(C)C)N(C)C(=O)CCCCCN1C(=O)C=CC1=O)C(C)C]

and then deprotect the group under acidic conditions to obtain the compound

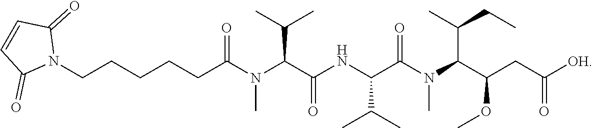 [SMILES: CC[C@H](C)[C@@H]([C@@H](CC(=O)O)OC)N(C)C(=O)[C@@H](NC(=O)[C@H](C(C)C)N(C)C(=O)CCCCCN1C(=O)C=CC1=O)C(C)C]

The concentration of trifluoroacetic acid ranges from 30% to 50%, preferably 35%. The deprotection reaction will be completed quickly, and then the reaction will be quenched immediately, and finally the yield of compound D can be increased from 5% to 50%.

### Example 1

The reaction route of this example is as follows:

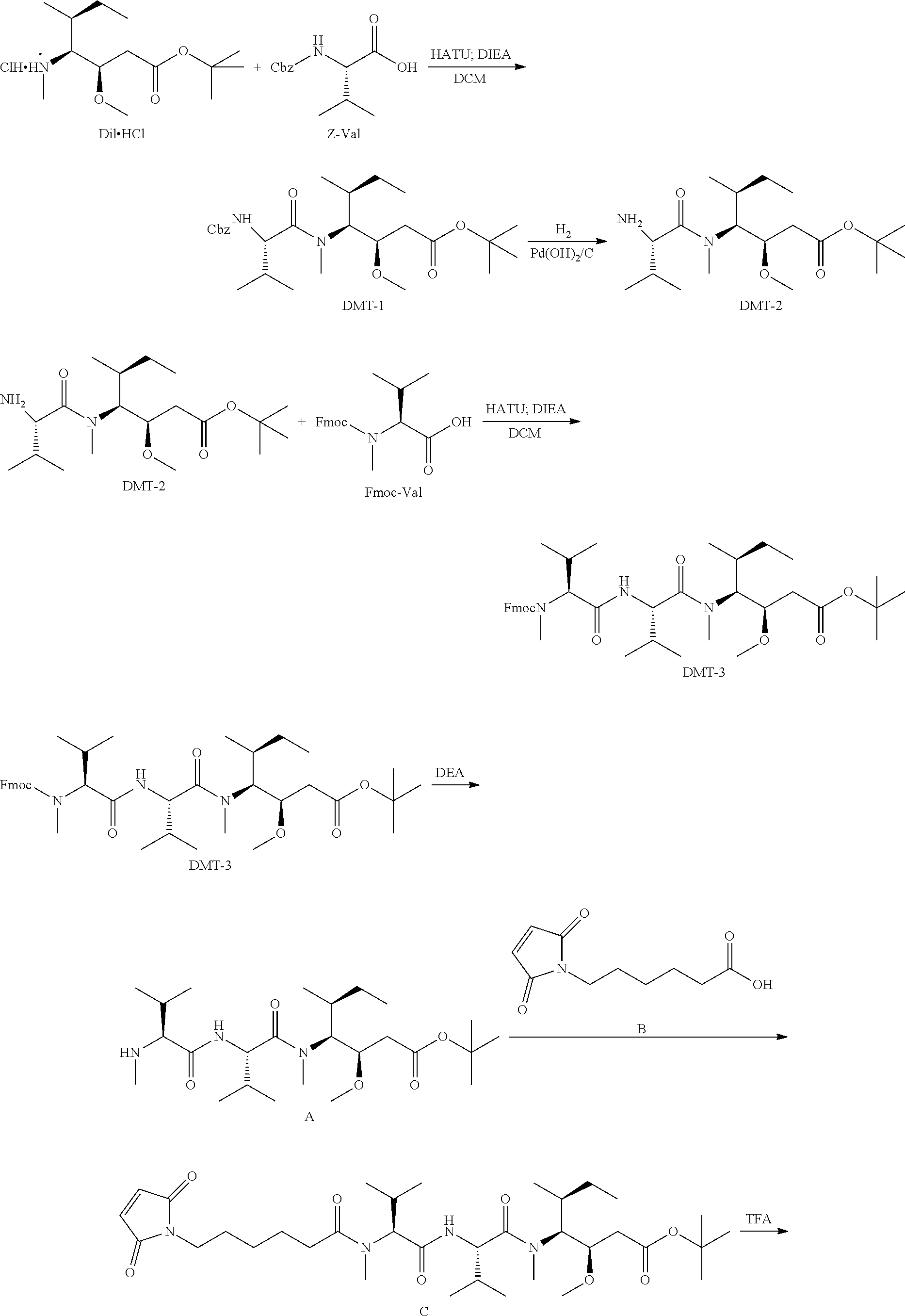

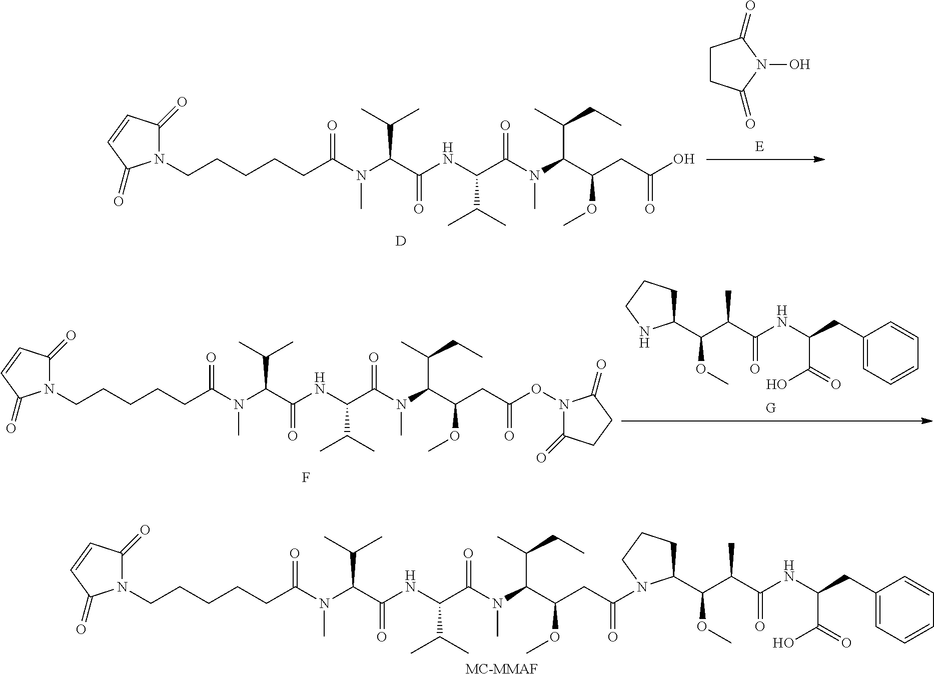

In a 3 L three-necked flask, add 1.5 L of dichloromethane and Dil.HCl (202.3 g, 0.683 mol 1.0 eq), magnetic stirring, nitrogen protection, then add Z-Val-OH (163.23 g, 0.65 mol, 0.95 eq) and HATU (311.6 g, 0.82 mol, 1.20 eq), stir at room temperature for 30 minutes, then cool to an ice bath, control the temperature at 10 degree and add DIEA (452.5 ml, 4.0 eq) dropwise, after the addition is completed, stir under ice bath for 30 minutes, move to room temperature, react for 16 hours, and detect by HPLC. The main peak is the product peak (retention time 29.98 min). The reaction of the raw material Dil.HCl is complete and the reaction is complete. Wash the reaction solution with citric acid aqueous solution (2 L*1), saturated sodium bicarbonate solution (2 L*1), saturated brine (2 L*1), dry the organic layer with anhydrous sodium sulfate, filter with suction, desolventize and obtain 531 g of crude product. Dissolve the crude product in 800 ml methanol, add 1.1 ml (1 mol/L) dilute hydrochloric acid (about 1 hour) dropwise with stirring in an ice bath, and stir at room temperature for 12 h. Stop stirring, separate the layers, separate the upper water layer and the lower layer. Dry the products by oil pump, and obtain 325 g DMT-1, and the yield is 91%.

Add 800 ml methanol and DMT-1 (LN114-38,325 g, 0.66 mol) and 110 g Pd(OH)2/C in a 2 L single-neck flask, replace with H2 three times, react at room temperature for 5 h, TLC monitors the raw material DMT-1 is complete reacted. Add diatomaceous earth to the sand core funnel, filter with suction, and wash the filter cake with 1 L methanol, collect the filtrate, evaporate the filtrate, and pump until the product does not foam, obtain 230.2 g of DMT-2, with a purity of 94%; yield: 97%.

In a 3 L three-neck flask, dissolve DMT-2 (LN114-40-01, 230.2 g, actual 0.60 mol, 1.0 eq) in 500 ml DCM, stir well, add Fmoc-Me-val (202.6 g, 0.57 mol, 0.95 eq) and HATU (292.9 g, 0.77 mol, 1.20 eq), then add 1 L of DCM, stir at room temperature for 30 min, then cool to an ice bath, and add DIEA (212.7 ml, 2.0 eq) dropwise at 10 degree. After stirring under the bath for 30 min, move to room temperature and react for 16.0 h. HPLC detects the main peak as the product peak (retention time 36.00 min). The reaction of the raw material DMT-2 is complete and the reaction is over. Wash the reaction solution with water (2.0 L*1), citric acid aqueous solution (2 L*1), saturated sodium bicarbonate solution (2 L*1), saturated brine (1 L*1), and wash the organic layer w with water. After the aqueous sodium sulfate is dried, filter with suction to remove the solvent to obtain the crude product 655 g. Dissolve the crude product in 650 ml methanol, and add 360 ml (1 mol/L) dilute hydrochloric acid dropwise with stirring. Stir at room temperature for 12 h, stop stirring, separate the layers, and separate the upper layer of water, so twice. Dry the lower product with an oil pump to obtain 373 g of DMT-3 with a purity of 96.7% by HPLC and a yield of 90%.

Add compound DMT-3 (5.0 g, 7.22 mmol) and diethylamine (5 mL) to dichloromethane (20 mL), stir and react at room temperature under nitrogen protection for 4 hours. LCMS shows that regard the compound DMT-3 in the reaction solution less than 3% as the end of the reaction. Spin-dry the reaction solution, and purify the crude product by medium pressure reverse phase (using 220 g industrial packed C18 reverse phase column), and purified gradient water/acetonitrile (90/10-10/90, v/v) for 1 hour. Collect the pure product and lyophilize to obtain a white solid compound A (white solid, 3.15 g, yield 93%). MS: 472.26 (M+H⁺).

Add compound B (1.77 g, 8.04 mmol), HATU (3.82 g, 10.05 mmol) and DIEA (1.72 g, 13.4 mmol) to dichloromethane (50 mL), stir and react at room temperature under nitrogen for 30 minutes, and then add the compound A (3.15 g, 6.68 mmol), stir and react at room temperature for 4 hours under the protection of nitrogen, LCMS shows that regard the compound A in the reaction solution less than 3% as the end of the reaction. Wash the reaction solution with citric acid aqueous solution (50 mL), saturated brine (50 mL), dry with anhydrous sodium sulfate and spin-dry. Purify the crude product by medium pressure reverse phase (use 120 g industrial packed C18 reverse phase column), and purified gradient water/Acetonitrile (90/10-10/90, v/v), time 1 hour. Collect the pure product and lyophilize to obtain a white solid compound C (white solid, 3.86 g, yield 87%). MS: 665.37 (M+H⁺)

Dissolve compound C (3.86 g, 5.81 mmol) in a mixed solvent of dichloromethane and trifluoroacetic acid (20 mL, 2/1, v/v), stir and the react at room temperature under nitrogen for 20 minutes. LCMS shows the compound in the reaction solution C is less than 5% as the end of the reaction. Dilute the reaction solution with 40 mL acetonitrile, concentrate at low temperature to about 10 mL volume, and purify by medium pressure reverse phase (using 220 g industrial packed C18 reverse phase column), and purified gradient water/acetonitrile (90/10-10/90, v/v)), the time is 2 hours. Collect the pure product and lyophilize to obtain a white solid compound D (1.59 g, yield 45%). MS: 609.30 (M+H⁺)

Dissolve compound D (1.59 g, 2.61 mmol), compound E (0.36 g, 3.13 mmol) and compound EDCI (0.60 g, 3.13 mmol) in dichloromethane (20 mL), and t stir and react at room temperature for 2 hours under nitrogen protection. LCMS shows when the compound D in the reaction solution is less than 5%, the reaction is deemed to be complete. Wash the reaction solution with saturated brine (20 mL), dry with anhydrous sodium sulfate, and spin-dry. Purify the crude product by medium-pressure reverse phase (40 g industrial packed C18 reverse phase column), and purified gradient water/acetonitrile (90/10-10)/90, v/v), the time is 1 hour. Collect the pure product and lyophilize to obtain a white solid compound F (white solid, 1.79 g, yield 97%). MS: 706.32 (M+H⁺)

Dissolve compound F (1.79 g, 2.53 mmol), compound G (0.93 g, 2.78 mmol) and DIEA (0.72 g, 5.56 mmol) in dichloromethane (20 mL), stir at room temperature and the reaction is under nitrogen protection for 18 hours. LCMS showed the compound F in the reaction liquid is less than 3%, the reaction is deemed to be complete. Wash the reaction solution with citric acid aqueous solution (20 mL) and saturated brine (20 mL) successively, dry over anhydrous sodium sulfate, and spin-dry. The crude product is purified by medium pressure reverse phase (Use 80 g industrial packed C18 reverse phase column), and pure gradient water/Acetonitrile (90/10-10/90, v/v), time 1 hour. Collect the pure product and lyophilize to obtain a white solid compound MC-MMAF (white solid, 2.01 g, yield 86%, HPLC purity 99% by UV 220 nm). MS: 925.66 (M+H⁺)

1. An intermediate compound for synthesizing MC-MMAF, its structural formula is: 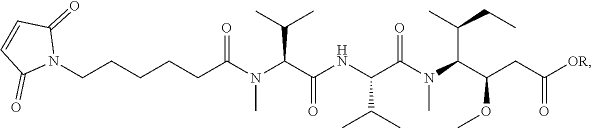
wherein, R is selected from a group consisting of hydrogen, succinimidyl, pentafluorophenyl, p-nitrophenyl, phthalamide and a mixture thereof.

2. A method for synthesizing MC-MMAF, wherein, the method is to perform a condensation reaction on a compound with structural formula 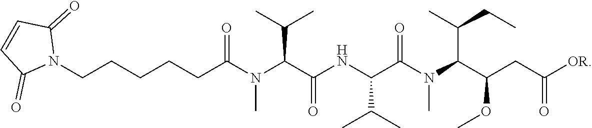
and a compound of structural formula 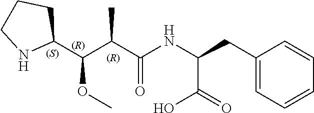
in a solvent.

3. (canceled)

4. (canceled)

5. (canceled)

6. (canceled)

7. (canceled)

8. (canceled)

9. (canceled)

10. (canceled)

11. The method according to claim 2, wherein the R is hydrogen, during the reaction, adding regent N in the presence of reagent M,
the reagent M is selected from a group consisting of DCC, DCEP, EDC, DIC, HATU, HBTU, HBPIPU, HBPyU, HSPyU, HCTU, HOTU, HOTT, HSTU, HDMA, TATU, TBTU, TCTU, TCFH, TDBTU, TOTU, TOTT, TPTU, TFFH, BTFFH, TNTU, TSTU, COMU, T3P, BOP, PyBOP, PyBrOP, PyClOP, Brop, PyAOP, PyCIU, CDI, TPSI, TSTU, DEPBT, DMTMM, EEDQ, CIP, CIB, DMC, HOAt, HOBt, EDCI and a mixture thereof,
the reagent N is selected from a group consisting of triethylamine, diisopropylethylamine (DIEA), pyridine, N, N-dimethyl-4-pyridine, and a mixture thereof;
the solvent is selected from a group consisting, of dichloromethane, dimethyl sulfoxide, N,N-dimethylformamide, N,N-dimethylacetamide, tetrahydrofuran, 1,4-dioxane and 2-methyl tetrahydrofuran and a mixture thereof; and
reaction temperature is 20° C. below zero to 40° C.

12. The method according to claim 11, wherein the reagent M is a mixture of EDCI and HOBt, and the reagent N is diisopropylethylamine (DIEA).

13. The method according to claim 11, wherein the reaction temperature is 10° C. below zero to 25° C.

14. The method according to claim 2, wherein the R is selected from a group consisting of succinimidyl, pentafluorophenyl, p-nitrophenyl, sand phthalmide, and reacts in the presence of reagent P, which is selected from a group consisting of ethylamine, diisopropylethylamine (DIEA), pyridine, N,N-dimethyl-4-pyridine, sodium carbonate, sodium bicarbonate, potassium carbonate, potassium bicarbonate, lithium carbonate and lithium bicarbonate;
the solvent is selected from a group consisting of dichloromethane, dimethyl sulfoxide, N,N-dimethylformamide, N,N-dimethylacetamide, tetrahydrofuran, 1,4-dioxane and 2-methyl tetrahydrofuran and a mixture thereof;
reaction temperature is 0° C. to 100° C.

15. The method according to claim 14, wherein the reagent P is sodium carbonate or diisopropylethylamine (DIEA).

16. The method according to claim 14, wherein the reaction temperature is 15° C. to 50° C.

17. The method according to claim 2, wherein, after the reaction is completed, separate MC-MMAF from the reaction solution.

18. The method according to claim 17, wherein the separation operation comprises evaporating the solvent under reduced pressure, and then purifying or recrystallization by medium pressure chromatography.

19. A method of synthesizing 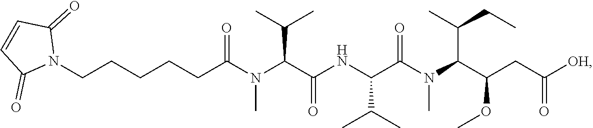
wherein, use a polypeptide 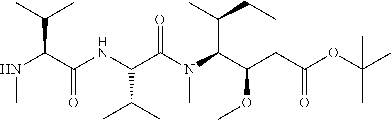
with a protective group first to react with 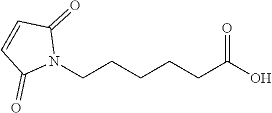
to synthesize a compound 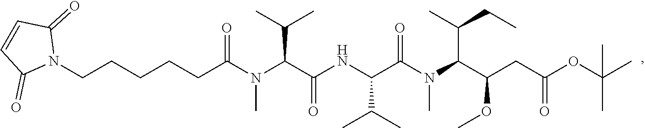 [SMILES: CC[C@H](C)[C@@H]([C@@H](CC(=O)OC(C)(C)C)OC)N(C)C(=O)[C@@H](NC(=O)[C@H](C(C)C)N(C)C(=O)CCCCCN1C(=O)C=CC1=O)C(C)C]
and then the protective group is deprotected under acidic conditions to obtain the compound 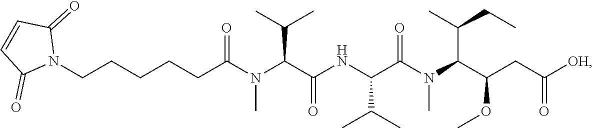 [SMILES: CC[C@H](C)[C@@H]([C@@H](CC(=O)O)OC)N(C)C(=O)[C@@H](NC(=O)[C@H](C(C)C)N(C)C(=O)CCCCCN1C(=O)C=CC1=O)C(C)C]
wherein trifluoroacetic acid is applicable to provide acidic conditions, and the acid concentration range is 30% to 50%.
